(19) United States
(12) Patent Application Publication
(10) Pub. No.: US 2023/0092005 A1
(43) Pub. Date: Mar. 23, 2023

(54) COMPOSITIONS AND METHODS FOR TREATING CANCER AND TUMOR

(71) Applicant: OBLATO, INC., Princeton, NJ (US)

(72) Inventors: Sin Wook KANG, Seongnam-si, Gyeonggi-do (KR); Kyeongsoon KIM, Busan (KR); Ji Hye SUNG, Seoul (KR)

(73) Assignee: OBLATO, INC., Princeton, NJ (US)

(21) Appl. No.: 17/801,839

(22) PCT Filed: Feb. 24, 2021
(86) PCT No.: PCT/US2021/019457

§ 371 Date: Aug. 24, 2022

Related U.S. Application Data
(60) Provisional application No. 62/980,645, filed on Feb. 24, 2020.

(51) Int. Cl. A61K 31/185; A61K 45/06; A61P 35/04

(52) U.S. Cl. CPC A61K 31/185; A61K 45/06; A61P 35/04

(57) ABSTRACT

Methods of treating a cancer or a tumor in a subject using a composition are provided. The composition includes a therapeutically effective amount of 2,4-disulfonyl a-phenyl tert-butyl nitrone (2,4-ds-PBN) or pharmaceutically acceptable salts thereof, wherein the therapeutically effective amount decreases a growth of the cancer or the tumor.

### CROSS-REFERENCE TO RELATED APPLICATIONS

This application claims priority to U.S. Provisional Patent Application No. 62/980,645, which was filed on Feb. 24, 2020, the entire contents of which are incorporated by reference herein.

### FIELD OF THE INVENTION

The presently disclosed subject matter relates to techniques for inhibiting the development of cancers and tumors. Specifically, the presently disclosed subject matter relates to methods and compositions for inhibiting the development of cancers and tumors.

### BACKGROUND

Cancer is a group of diseases involving abnormal growth of cells that can have the potential to invade other parts/tissues of a subject, causing severe malfunctions. For example, lung cancer is a type of cancer that starts abnormal cell growth in the lungs. There are two main types of lung cancer that can be treated differently. The first type is non-small cell lung cancer (NSCLC). NSCLC grows slowly and causes relatively fewer symptoms until it has advanced. Subtypes of NSCLC can include adenocarcinoma, squamous cell carcinoma, adenosquamous carcinoma, sarcomatoid carcinoma, and large cell carcinoma. The second type is small cell lung cancer (SCLC). SCLC can grow and spread to other tissues than faster NSCLC. Due to its fast growth rates, chemotherapy and radiation can be applied to treat SCLS. Although NSCLC can be less aggressive, NCLC can be diagnosed at later stages, as NSCLC cases fewer symptoms until it has advanced, leading to a fatal prognosis.

Cancer cells can grow uncontrollably and form a mass called a tumor. Cancer is one of the leading causes of death and is responsible for about 1 in 6 deaths globally. Cancer and/or tumor formation can be the result of the interaction between genetic factors of a subject and external factors (e.g., physical carcinogens, chemical carcinogens, and biological carcinogens). Despite advances in the understanding of molecular mechanisms of cancer, there are certain side effects in currently available anti-cancer and/or anti-tumor therapies. Although 2,4-disulfonyl a-phenyl tert-butyl nitrone (2,4-ds-PBN) can suppress glioblastoma (GBM) tumor growth in rodent glioma model and increase the sensitivity of other anti-cancer drugs for the GBM treatment, its therapeutic effects on the treatment of other tumors and/or cancers have not been identified.

Therefore, there is a need for improved techniques for treating cancer or tumor (e.g., NSCLC) using 2,4-ds-PBN.

### SUMMARY

The present disclosure provides pharmaceutical compositions and methods for treating a cancer or a tumor. The method for treating a cancer or a tumor in a subject can include administering a composition that comprises a therapeutically effective amount of 2,4-disulfonyl a-phenyl tert-butyl nitrone (2,4-ds-PBN) or pharmaceutically acceptable salts thereof, wherein the therapeutically effective amount can decrease a growth of the cancer or the tumor.

In certain embodiments, the cancer can be a non-small cell lung carcinoma.

In certain embodiments, the therapeutically effective amount of the 2,4-ds-PBN or pharmaceutically acceptable salts thereof can be from about 5 mg/kg body weight/day to about 1,000 mg/kg body weight/day.

In certain embodiments, the therapeutically effective amount of the 2,4-ds-PBN or pharmaceutically acceptable salts thereof can be delivered to the subject via an intravenous injection or an intra-arterial injection. In non-limiting embodiments, the therapeutically effective amount of the 2,4-ds-PBN or pharmaceutically acceptable salts thereof can be delivered to the subject via an enteral administration or an oral administration.

In certain embodiments, the method can further include measuring an area, a volume, or a combination thereof of the cancer or the tumor. In non-limiting embodiments, the cancer volume or the tumor volume decreases by at least about 50% after administrating the therapeutically effective amount of the 2,4-ds-PBN or pharmaceutically acceptable salts thereof. In non-limiting embodiments, a growth of the cancer or the tumor decreases by at least about 40% after administrating the therapeutically effective amount of the 2,4-ds-PBN or pharmaceutically acceptable salts thereof.

In certain embodiments, the method can further include counting apoptotic cancer cells or apoptotic tumor cells. In non-limiting embodiments, a number of the apoptotic cancer cells or the apoptotic tumor cells increases by at least about 15% after administrating the therapeutically effective amount of the 2,4-ds-PBN or pharmaceutically acceptable salts thereof.

In certain embodiments, the therapeutically effective amount of the 2,4-ds-PBN or pharmaceutically acceptable salts thereof can be administered to the subject at least once a week.

In certain embodiments, the method can further include administering a therapeutically effective amount of an anti-cancer agent or an anti-tumor agent to the subject. In non-limiting embodiments, the anti-cancer/tumor agent can be selected from the group consisting of a chemotherapeutic agent, a radiotherapeutic agent, a cytokine, an anti-angiogenic agent, a tyrosine kinase inhibitor (TKI), an apoptosis-inducing agent, an anti-cancer antibody, an immunotherapeutic agent, and combinations thereof.

In certain embodiments, the method can further include administering an additional therapy. The additional therapy can be selected from the group consisting of an anti-tumor therapy, an anti-cancer therapy, a chemotherapy, a targeted therapy, an immunotherapy, a radiation therapy, a radiofrequency ablation therapy, surgery, a therapy using a tumor treating fields (TTFields) device, or combinations thereof.

In certain embodiments, the present disclosure provides a pharmaceutical composition for treating a cancer or a tumor in a subject that includes a therapeutically effective amount of 2,4-disulfonyl a-phenyl tert-butyl nitrone (2,4-ds-PBN) or pharmaceutically acceptable salts thereof, wherein the therapeutically effective amount can be present in an amount to decrease a growth of the cancer or the tumor.

In certain embodiments, the pharmaceutical composition can be in a form of a tablet, a pill, a capsule, a gel, a liquid, a syrup, a slurry, or a suspension for an oral administration or an enteral administration. In non-limiting embodiments, the therapeutically effective amount of the 2,4-ds-PBN or pharmaceutically acceptable salts thereof for the oral administration or the enteral administration can be from about 5 mg/kg body weight/day to about 1,000 mg/kg body weight/day.

In certain embodiments, the pharmaceutical composition can be in a form of a solution or a liquid for an intravenous injection or an intra-arterial injection. In non-limiting embodiments, the therapeutically effective amount of the 2,4-ds-PBN or pharmaceutically acceptable salts thereof for the intravenous injection or the intra-arterial injection can be from about 10 mg/kg body weight/day to about 500 mg/kg body weight/day.

In certain embodiments, the cancer can be a non-small cell lung carcinoma.

### BRIEF DESCRIPTION OF THE DRAWINGS

FIG. 1 provides a graph showing tumor volumes after administering negative control, OKN-007 (150 mg/kg, 300 mg/kg, or 600 mg/kg), or cisplatin (2 mg/kg). Each point represents the mean values ±standard deviation (S.D.) of ten independent experiments. ## represents the p-value is less than 0.01 (p<0.01) compared with the negative control group by Steel's t-test. ** represents the p-value is less than 0.01 (p<0.01) compared with the negative control group by Dunnett's t-test.

FIG. 2 provides a graph showing tumor weights after administering negative control, OKN-007 (150 mg/kg, 300 mg/kg, or 600 mg/kg), or cisplatin (2 mg/kg). Each point represents the mean values ±standard deviation (S.D.) of ten independent experiments. ** represents the p-value is less than 0.01 (p<0.01) compared with the negative control group by Dunnett's t-test.

FIG. 3 provides photographs of A549 tumor-bearing nude mice at the end of the observation period after administering negative control, OKN-007 (150 mg/kg, 300 mg/kg, or 600 mg/kg), or cisplatin (2 mg/kg).

FIG. 4 provides photographs of tumors removed from A549 tumor-bearing nude mice at the end of the observation period after administering negative control or OKN-007 (150 mg/kg). In the negative control group (G1), the tumor size and volume increase over time. The OKN-007 (150 mg/kg) treated group (G2) shows a statistically significant decrease in tumor volume and size compared to the negative control group (G1).

FIG. 5 provides photographs of tumors removed from A549 tumor-bearing nude mice at the end of the observation period after administering negative OKN-007 (300 or 600 mg/kg). The OKN-007 (300 mg/kg) treated group (G3) and the OKN-007 (600 mg/kg) treated group (G4) show a statistically significant decrease in tumor volume and size compared to the negative control group (G1).

FIG. 6 provides photographs of tumors removed from A549 tumor-bearing nude mice at the end of the observation period after administering cisplatin (2 mg/kg). As a positive control, the cisplatin (2 mg/kg) treated group (G5) shows a statistically significant decrease in tumor volume and size compared to the negative control.

FIG. 7 provides hematoxylin and eosin (H&E) staining and terminal deoxynucleotidyl transferase dUTP nick end labeling (TUNEL) assay images of tumors harvested from the negative control-treated group (G1). Necrosis of the tumors was evaluated by the H&E staining images, and apoptosis of tumor cells was evaluated by the TUNEL assay images.

FIG. 8 provides H&E staining and TUNEL assay images of tumors harvested from the OKN-007 (150 mg/kg) treated group (G2). The OKN-007 (150 mg/kg) treated group (G2) shows a significant increase in the number of apoptotic cells (arrow) in the tumor compared to the negative control group (G1).

FIG. 9 provides H&E staining and TUNEL assay images of tumors harvested from the OKN-007 (300 mg/kg) treated group (G3). The OKN-007 (300 mg/kg) treated group (G3) shows a significant increase in the number of apoptotic cells (arrow) in the tumor compared to the negative control group (G1).

FIG. 10 provides H&E staining and TUNEL assay images of tumors harvested from the OKN-007 (600 mg/kg) treated group (G4). The OKN-007 (600 mg/kg) treated group (G4) shows a significant increase in the number of apoptotic cells (arrow) in the tumor compared to the negative control group (G1).

FIG. 11 provides H&E staining and TUNEL assay images of tumors harvested from the cisplatin (2 mg/kg) treated group (G5). As a positive control, the cisplatin (2 mg/kg) treated group (G5) shows a significant increase in the number of apoptotic cells (arrow) in the tumor compared to the negative control group (G1).

### DETAILED DESCRIPTION

The detailed description of the disclosed subject matter is divided into the following subsections for clarity and not by way of limitation:
I. Definitions;
II. Pharmaceutical Compositions; and
III. Methods of Use.

### I. Definitions

The terms used in this specification generally have their ordinary meanings in the art, within the context of the disclosed subject matter and in the specific context where each term is used. Certain terms are discussed below, or elsewhere in the specification, to provide additional guidance to the practitioner in describing the compositions and methods of the disclosed subject matter.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing,” and “comprising” are interchangeable, and one of skill in the art is cognizant that these terms are open-ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “administering” can mean any suitable route (e.g., systemic administration, oral administration, and intravenous administration).

The term “agent,” as used herein, means a substance that produces or is capable of producing an effect and would include, but is not limited to, chemicals, pharmaceuticals, biologics, small organic molecules, antibodies, nucleic acids, peptides, and proteins.

An “anti-cancer effect” or an “anti-tumor effect” refers to one or more of a reduction in aggregate cancer cell mass, a reduction in cancer cell growth rate, a reduction in cancer progression, a reduction in cancer cell proliferation, a reduction in tumor mass, a reduction in tumor volume, a reduction in tumor cell proliferation, a reduction in tumor growth rate and/or a reduction in tumor metastasis. In certain embodiments, an anti-cancer effect can refer to a complete response, a partial response, a stable disease (without progression or relapse), a response with a later relapse, or progression-free survival in a patient diagnosed with cancer.

An “anti-cancer agent” or an “anti-tumor agent” as used herein, can be any molecule, compound, chemical, or composition that has an anti-cancer effect. Anti-cancer agents include, but are not limited to, chemotherapeutic agents, radiotherapeutic agents, cytokines, anti-angiogenic agents, tyrosine kinase inhibitors (TKI), apoptosis-inducing agents, anti-cancer antibodies, and/or agents that promote the activity of the immune system. In certain embodiments, an anti-cancer agent can be a radiotherapeutic agent. In certain embodiments, an anti-cancer agent can be an immunotherapeutic agent. In certain embodiments, an anti-cancer agent can be a chemotherapeutic agent. Other non-limiting exemplary anti-cancer agents that can be used with the presently disclosed subject matter include tumor-antigen based vaccines and chimeric antigen receptor T-cells.

As used herein, the term “co-administer” is meant that a composition described herein is administered at the same time, just prior to, or just after the administration of additional therapies. The composition of the disclosure can be administered alone or can be co-administered with a second composition/therapeutic agent to a subject. Co-administration is meant to include simultaneous or sequential administration of the composition individually or in combination with a second composition/therapeutic agent. Additionally, the first and second agents can be formulated separately or together in one or more compositions.

As used herein, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “concurrent administration” includes overlapping in duration at least in part. For example, when two agents (e.g., any of the compositions described herein) are administered concurrently, their administration occurs within a certain desired time. The compositions' administration can begin and end on the same day. The administration of one composition can also precede the administration of a second composition by day(s) as long as both compositions are taken on the same day at least once. Similarly, the administration of one composition can extend beyond the administration of a second composition as long as both compositions are taken on the same day at least once. The compositions do not have to be taken at the same time each day to include concurrent administration.

As used herein, the term “disease” refers to any condition or disorder that damages or interferes with the normal function of a cell, tissue, or organ.

The terms “detection” or “detecting” include any means of detecting, including direct and indirect detection.

The term “dosage” is intended to encompass a formulation expressed in terms of total amounts for a given timeframe, for example, as μg/kg/hr, μg/kg/day, mg/kg/day, or mg/kg/hr. The dosage is the amount of an ingredient administered in accordance with a particular dosage regimen. A “dose” is an amount of an agent administered to a mammal in a unit volume or mass, e.g., an absolute unit dose expressed in mg of the agent. The dose depends on the concentration of the agent in the formulation, e.g., in moles per liter (M), mass per volume (m/v), or mass per mass (m/m). The two terms are closely related, as a particular dosage results from the regimen of administration of a dose or doses of the formulation. The particular meaning, in any case, will be apparent from the context.

An “effective amount” or “therapeutically effective amount” of an agent refers to an amount effective, at dosages and for periods of time necessary, to achieve the desired therapeutic or prophylactic result, e.g., treating cancer in a subject. A therapeutically effective amount can be administered in one or more administrations.

An “individual” or “subject” herein is a vertebrate, such as a human or non-human animal, for example, a mammal. Mammals include, but are not limited to, humans, primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

The terms “inhibiting,” “eliminating,” “decreasing,” “reducing,” or “preventing,” or any variation of these terms, referred to herein, include any measurable decrease or complete inhibition to achieve a desired result.

The term “in need thereof” would be a subject known or suspected of having or being at risk of developing a disease, e.g., cancer.

As used herein, “liquid” is a dosage form consisting of a composition in its liquid state. A liquid is pourable; it flows and conforms to its container at room temperature. Liquids display Newtonian or pseudoplastic flow behavior. In certain embodiments, a “semi-liquid” as used herein can have properties of both a liquid and another formulation (i.e., a suspension, an emulsion, a solution, a cream, a gel, a jelly, and the like).

As used herein, “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. The use of such media and agents for pharmaceutically active substances is well known in the art.

As used herein, the terms “prevent,” “preventing,” or “prevention,” “prophylactic treatment,” and the like refer to reducing the probability of developing a disorder or condition in a subject who does not have but is at risk of or susceptible to developing a disorder or condition. The prevention can be complete (i.e., no detectable symptoms) or partial so that fewer symptoms are observed than would likely occur absent treatment. The terms further include a prophylactic benefit. For disease or condition to be prevented, the compositions can be administered to a patient at risk of developing a particular disease or to a patient reporting one or more of the physiological symptoms of a disease, even though a diagnosis of this disease cannot have been made.

As used herein, a “solution” is a clear, homogeneous liquid dosage form that contains one or more chemical substances dissolved in a solvent or mixture of mutually miscible solvents. A solution is a liquid preparation that contains one or more dissolved chemical substances in a suitable solvent or mixture of mutually miscible solvents. Because molecules of a drug substance in solution are uniformly dispersed, the use of solutions as dosage forms generally provides assurance of uniform dosage upon administration and good accuracy when the solution is diluted or otherwise mixed.

As used herein, “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, prolonging survival, preventing recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the presently disclosed subject matter are used to delay the development of a disease or to slow the progression of a disease, e.g., cancer and/or tumor.

As used herein, “sequential administration” includes that the administration of two agents (e.g., compositions described herein) occurs separately on the same day or does not occur on the same day (e.g., occurs on consecutive days).

As described herein, any concentration range, percentage range, ratio range, or integer range is to be understood to include the value of any integer within the recited range and, when appropriate, fractions thereof (such as one-tenth and one-hundredth of an integer), unless otherwise indicated.

II. Pharmaceutical Compositions

The present disclosure provides pharmaceutical compositions comprising a therapeutically effective amount of 2,4-disulfonyl a-phenyl tert-butyl nitrone (2,4-ds-PBN) or pharmaceutically acceptable salts thereof to decrease a growth of the cancer or the tumor.

In certain embodiments, 2,4-ds-PBN can exist in the following acid form of Formula I as a solid or in solution in lower pH conditions:

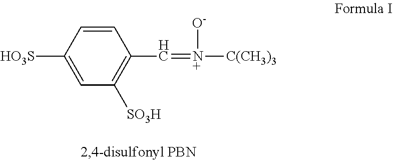 [SMILES: CC(C)(C)/[N+]([O-])=C\c1ccc(S(=O)(=O)O)cc1S(=O)(=O)O]

In non-limiting embodiments, 2,4-ds-PBN can exist in the following ionized salt form of Formula II or Formula III at higher pH conditions:

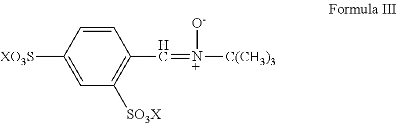

In some embodiments, X in Formula III can be a pharmaceutically acceptable cation. For example, this cation can be a monovalent material such as sodium, potassium, or ammonium. In non-limiting embodiments, the cation can be a multivalent alone or cation in combination with a pharmaceutically acceptable monovalent anion. For example, the pharmaceutically acceptable monovalent anion can include calcium with chloride, bromide, iodide, hydroxyl, nitrate, sulfonate, acetate, tartrate, oxalate, succinate, palmoate or the like anion, magnesium with such anions, zinc with such anions, or the like. In non-limiting embodiments, the free acid and the sodium, potassium, calcium, magnesium, or ammonium salts can be preferred.

In certain embodiments, the 2,4-ds-PBN can be prepared by a two-step reaction sequence. For example, tertiary butyl nitrate (2-methyl-2-nitropropane) can be converted to the corresponding n-hydroxylamine using a suitable catalyst such as an activated zinc/acetic acid catalyst or an aluminum/mercury amalgam catalyst. This reaction can be carried out in about 0.5 to 12 hours and especially about 2 to 6 hours or so at a temperature of about 15-100° C. in a liquid reaction medium such as alcohol/water mixture in the case of the zinc catalyst or an ether/water mixture in the case of the aluminum amalgam catalyst. The freshly formed hydroxylamine can be reacted with 4-formyl-1,3-benzenedisulfonic acid, with a slight excess of the amine being used. This reaction can be carried out at similar temperature conditions. This reaction can be completed in from about 10 to about 24 hours. The formed material through the two reactions can be the free acid and be characterized by a molecular weight of 337.3 g/mole. In some embodiments, the formed material can be a white powdery material that decomposes upon heating. In non-limiting embodiments, the formed material can be characterized by solubility in water of greater than 1 gram/ml and a¹H NMR spectrum in D₂O of 8.048 ppm (dd, 8.4, 1.7 Hz); 8.836 ppm (d, 8.4 Hz); 8.839 ppm (d, 1.7 Hz); 8.774 ppm (s).

The various salts can be easily formed by admixing the free acid in a medium with a base, for example, KOH for potassium salt, and the like.

In certain embodiments, the disclosed composition can have improved transport efficiency. For example, the 2,4-ds-PBN can provide improved transport efficiency across the blood/brain barrier.

In certain embodiments, the pharmaceutical compositions suitable for use in the presently disclosed subject matter can include compositions where the active ingredients, e.g., 2,4-ds-PBN, are contained in a therapeutically effective amount. The therapeutically effective amount of an active ingredient can vary depending on the active ingredient, compositions used, cancer and/or tumor and its severity, and the age, weight, etc., of the subject to be treated. In certain embodiments, a subject can receive a therapeutically effective amount of the disclosed composition in single or multiple administrations of one or more composition, which can depend on the dosage and frequency as required and tolerated by the patient.

In certain embodiments, the therapeutically effective amount of 2,4-ds-PBN or pharmaceutically acceptable salts thereof in the pharmaceutical composition can be at least about 5 mg/kg body weight/day, at least about 10 mg/kg body weight/day, at least about 20 mg/kg body weight/day, at least about 50 mg/kg body weight/day, at least about 100 mg/kg body weight/day, at least about 200 mg/kg body weight/day, at least about 300 mg/kg body weight/day, at least about 400 mg/kg body weight/day, at least about 500 mg/kg body weight/day, at least about 600 mg/kg body weight/day, at least about 700 mg/kg body weight/day, at least about 800 mg/kg body weight/day, at least about 900 mg/kg body weight/day, or at least about 1,000 mg/kg body weight/day. In non-limiting embodiments, the effective dose can be from about 5 mg/kg body weight/day to about 1,000 mg/kg body weight/day, from about 10 mg/kg body weight/day to about 1,000 mg/kg body weight/day, from about 10 mg/kg body weight/day to about 400 mg/kg body weight/day, from about 10 mg/kg body weight/day to about 300 mg/kg body weight/day, from about 10 mg/kg body weight/day to about 200 mg/kg body weight/day, from about 10 mg/kg body weight/day to about 100 mg/kg body weight/day, from about 20 mg/kg body weight/day to about 1,000 mg/kg body weight/day, from about 50 mg/kg body weight/day to about 1,000 mg/kg body weight/day, from about 50 mg/kg body weight/day to about 600 mg/kg body weight/day, from about 50 mg/kg body weight/day to about 400 mg/kg body weight/day, from about 50 mg/kg body weight/day to about 200 mg/kg body weight/day, from about 60 mg/kg body weight/day to about 1,000 mg/kg body weight/day, from about 70 mg/kg body weight/day to about 1,000 mg/kg body weight/day, from about 80 mg/kg body weight/day to about 1,000 mg/kg body weight/day, from about 90 mg/kg body weight/day to about 1,000 mg/kg body weight/day, from about 100 mg/kg body weight/day to about 1,000 mg/kg body weight/day, from about 200 mg/kg body weight/day to about 1,000 mg/kg body weight/day, from about 300 mg/kg body weight/day to about 1,000 mg/kg body weight/day, from about 400 mg/kg body weight/day to about 1,000 mg/kg body weight/day, from about 500 mg/kg body weight/day to about 1,000 mg/kg body weight/day, from about 600 mg/kg body weight/day to about 1,000 mg/kg body weight/day, from about 700 mg/kg body weight/day to about 1,000 mg/kg body weight/day, from about 800 mg/kg body weight/day to about 1,000 mg/kg body weight/day, from about 5 mg/kg body weight/day to about 900 mg/kg body weight/day, from about 5 mg/kg body weight/day to about 800 mg/kg body weight/day, from about 5 mg/kg body weight/day to about 700 mg/kg body weight/day, from about 5 mg/kg body weight/day to about 600 mg/kg body weight/day, from about 5 mg/kg body weight/day to about 500 mg/kg body weight/day, from about 5 mg/kg body weight/day to about 400 mg/kg body weight/day, from about 5 mg/kg body weight/day to about 300 mg/kg body weight/day, from about 5 mg/kg body weight/day to about 200 mg/kg body weight/day, or from about 5 mg/kg body weight/day to about 100 mg/kg body weight/day.

The pharmaceutical composition containing the active compound (e.g., 2,4-ds-PBN) can contain a physiologically compatible vehicle, as those skilled in the art can select using conventional criteria. Suitable pharmaceutically acceptable vehicles that can be used with the presently disclosed subject matter have the characteristics of not interfering with the effectiveness of the biological activity of the active ingredients, e.g., 2,4-ds-PBN, and that is not toxic to the patient to whom it is administered. In non-limiting embodiments, suitable pharmaceutical vehicles can include phosphate-buffered saline solutions, water, emulsions, such as oil/water emulsions, various types of wetting agents, and sterile solutions. Additional non-limiting examples of pharmaceutically acceptable vehicles include gels, bioabsorbable matrix materials, implantation elements containing the inhibitor and/or any other suitable vehicle, delivery, or dispensing means or material. Such pharmaceutically acceptable vehicles can be formulated by conventional methods and can be administered to the subject. In certain embodiments, the pharmaceutical acceptable vehicles can include buffers such as phosphate, citrate, and other organic acids; antioxidants including ascorbic acid and methionine; preservatives (such as, but not limited to, octadecyl dimethyl benzyl ammonium chloride, hexamethonium chloride, benzalkonium chloride, benzethonium chloride, butyl or benzyl alcohol, alkyl parabens such as methyl or propylparaben, catechol, resorcinol, cyclohexanol, 3-pentanol and m-cresol); low molecular weight (less than about 10 residues) polypeptides; proteins, such as serum albumin, gelatin or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). In certain embodiments, the suitable pharmaceutically acceptable vehicles can include one or more of water, saline, phosphate-buffered saline, dextrose, glycerol, ethanol, or combinations thereof.

In certain embodiments, the pharmaceutical composition can be formulated for oral administration. For example, the pharmaceutical compositions of the present disclosure can be formulated using pharmaceutically acceptable carriers well known in the art that are suitable for oral administration. Such carriers enable the pharmaceutical compositions to be formulated as tablets, pills, capsules, liquids, gels, syrups, slurries, suspensions, and the like, for oral or nasal ingestion by a patient to be treated. In non-limiting embodiments, the pharmaceutical composition can be a solid dosage form. In some embodiments, the tablet can be an immediate-release tablet. Alternatively or additionally, the tablet can be an extended or controlled release tablet. The solid dosage can include both an immediate release portion and an extended or controlled release portion.

In certain embodiments, the pharmaceutical composition can be formulated as an enteric-coated tablet or an enteric-coated capsule. For example, 2,4-ds-PBN can be enterically coated with at least one coating layer. In non-limiting embodiments, a coating material for enteric coating can include a delayed-release enteric polymers. Suitable examples of delayed-release enteric polymers can include, but are not limited to cellulose acetate phthalate, cellulose acetate trimellitate, hydroxypropyl methylcellulose phthalate, polyvinyl acetate phthalate, carboxymethylethylcellulose, co-polymerized methacrylic acid/methacrylic acid methyl esters such as, for instance, materials known under the trade name EUDRAGIT® L12.5, L100, EUDRAGIT® S12.5, S100, or similar compounds used to obtain enteric coatings. Co-polymerized methacrylic acid/methacrylic acid methyl esters can include three subclasses of compound: methacrylic acid copolymer type A, methacrylic acid copolymer type B, and methacrylic acid copolymer type C. The various types of copolymers represent compounds with varying ratios of methacrylic acid to methacrylic acid methyl ester. For example, methacrylic acid copolymer type A has a ratio of methacrylic acid to methacrylic acid methyl ester of approximately 1:1, type B has a ratio of approximately 1:2, and type C has a ratio similar to type A, but can incorporate additional components, such as surfactants. Aqueous colloidal polymer dispersions or re-dispersions can also be applied, including, for example, the polymers sold under the trade name EUDRAGIT® L 30D-55, EUDRAGIT® L100-55, EUDRAGIT® S100, EUDRAGIT® preparation 4110D (Rohm Pharma); EUDAGRITO FS 30D; AQUATERIC®, AQUACOAT® CPD 30 (FMC); KOLLICOAT MAE® 30D and 30DP (BASF); and EASTACRYL® 30D (Eastman Chemical). In some embodiments, the delayed-release enteric polymer can include methacrylic acid copolymer type A. In non-limiting embodiments, the delayed-release enteric polymer can include a mixture of methacrylic acid copolymer type A and methacrylic acid copolymer type B. One skilled in the art will appreciate that additional components can be added to the delayed-release polymers without departing from the scope of the disclosure. For example, a plasticizer can be added to the delayed-release enteric polymers to improve the physical characteristics of the delayed-release polymeric layer. Non-limiting examples of plasticizers can include triethyl citrate, acetyl triethyl citrate, tributyl citrate, acetyl tributyl citrate, trihexyl citrate, acetyl trihexyl citrate, trioctyl citrate, acetyl trioctyl citrate, butyryl trihexyl citrate, acetyl butyryl trihexyl citrate, trimethyl citrate, acetylated monoglycerides, alkyl sulphonic acid phenyl esters, or combinations thereof. In some embodiments, the plasticizer can include triethyl citrate.

In certain embodiments, the disclosed enteric polymers can be modified by mixing with other known coating products that are not pH sensitive. Examples of such coating products can include the neutral methacrylic acid esters with a small portion of trimethylammonioethyl methacrylate chloride, sold currently under the trade names EUDRAGIT® and EUDRAGIT® RL; a neutral ester dispersion without any functional groups, sold under the trade names EUDRAGIT® NE30D and EUDRAGIT® NE30; and other pH-independent coating products.

In certain embodiments, an additional modifying layer can be added on top of the enteric coating layer. This modifying layer can include a water penetration barrier layer (semipermeable polymer), which can be successively coated after the enteric coating to reduce the water penetration rate through the enteric coating layer and thus increase the lag time of the drug release. Controlled-release coatings known to one skilled in the art can be used for this purpose by coating techniques such as pan coating or fluid bed coating using solutions of polymers in water or suitable organic solvents or by using aqueous polymer dispersions. For example, the following non-limiting list of controlled release polymers can be used in the current disclosure: cellulose acetate, cellulose acetate butyrate, cellulose acetate propionate, ethylcellulose, hydroxypropyl methylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(propylene), poly(ethylene oxide), poly(ethylene terephthalate), poly(vinyl isobutyl ether), poly(vinyl acetate), poly(vinyl chloride), polyurethane, ethylcellulose aqueous dispersions such as AQUACOAT® and SURELEASE®, poly(butyl methacrylate, (2-dimethylaminoethyl) methacrylate, methyl methacrylate), poly(methacrylic acid, methyl methacrylate), poly(methacrylic acid, ethylacrylate), poly(methyl acrylate, methyl methacrylate, methacrylic acid), poly(ethylacrylate, methylmethacrylate, trimethylammonioethyl methacrylate chloride), poly(ethylacrylate, methyl methacrylate), poly(methacrylic acid, ethylacrylate), type A methacrylic acid copolymer, type B methacrylic acid copolymer, type C methacrylic acid copolymer, methacrylic acid copolymer dispersion, aqueous acrylic polymer dispersion, (EUDRAGIT® compounds), OPADRY®, fatty acids and their esters, waxes, zein, and aqueous polymer dispersions such as EUDRAGIT® RS and RL 30D, EUDRAGIT® NE 30D, cellulose acetate latex. The combination of the above polymers and hydrophilic polymers such as hydroxyethyl cellulose, hydroxypropyl cellulose (KLUCEL®, Hercules Corp.), hydroxypropyl methylcellulose (METHOCEL®, Dow Chemical Corp.), and polyvinylpyrrolidone can be incorporated. In non-limiting embodiments, the controlled release polymer can include ethylcellulose, hydroxypropyl methylcellulose, and combinations thereof. In some embodiments, the controlled release polymer can include a combination of ethylcellulose and hydroxypropyl methylcellulose in a ratio of ethylcellulose to hydroxypropyl methylcellulose ranging from about 0.1 to about 10, from about 0.2 to about 5, from about 0.5 to about 3, and from about 1 to about 2. In non-limiting embodiments, the controlled release polymer can include a combination of ethylcellulose aqueous dispersion and hydroxypropyl methylcellulose in a ratio of ethylcellulose aqueous dispersion to hydroxypropyl methylcellulose ranging from about 0.1 to about 10, from about 0.1 to about 5, from about 0.5 to about 4, and from about 1.5 to about 3.

In certain embodiments, the coating layer can solubilize at a pH of greater than about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1 or 7.2, and values or ranges therein between in a physiological environment. In non-limiting embodiments, the coating layer can prevent the release of 2,4-ds-PBN at a pH of less than about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1 or 7.2, and values or ranges therein between in a physiological environment. The physiological environment can be replicated, for example, by following the requirements of the United States Pharmacopeia (USP) disintegration test for enteric-coated tablets.

In certain embodiments, the coating to the preparations can be achieved by coating techniques known in the art (e.g., spraying, fluidized bed, immersion tube, or immersion bed techniques).

In non-limiting embodiments, the pharmaceutical compositions of the present disclosure can be formulated using pharmaceutically acceptable carriers well known in the art that are suitable for parenteral administration. The terms “parenteral administration” and “administered parenterally,” as used herein, refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intra sternal injection and infusion. For example, and not by way of limitation, pharmaceutical compositions of the present disclosure can be administered to the patient intravenously in a pharmaceutically acceptable carrier such as physiological saline. In certain embodiments, the present disclosure provides a parenteral pharmaceutical composition comprising inhibitors disclosed herein.

In non-limiting embodiments, the disclosed composition can include a a therapeutically effective amount of 2,4-ds-PBN or pharmaceutically acceptable salts thereof, wherein the therapeutically effective amount can be present in an amount to decrease a growth of the cancer or the tumor, and wherein the cancer can be a non-small cell lung carcinoma, wherein the pharmaceutical composition can be in a form of a tablet, a pill, a capsule, a gel, a liquid, a syrup, a slurry, or a suspension for oral administration or an enteral administration, wherein the therapeutically effective amount of the 2,4-ds-PBN or pharmaceutically acceptable salts thereof for oral administration or the enteral administration can be from about 5 mg/kg body weight/day to about 1,000 mg/kg body weight/day, wherein the composition can include at least one coating layer that can encapsulate the 2,4-ds-PBN or pharmaceutically acceptable salts thereof and solubilize at a predetermined range of pH, or alternatively wherein the pharmaceutical composition can be in a form of a solution or a liquid for an intravenous injection or an intra-arterial injection, and wherein the therapeutically effective amount of the 2,4-ds-PBN or pharmaceutically acceptable salts thereof for the intravenous injection or the intra-arterial injection can be from about 10 mg/kg body weight/day to about 500 mg/kg body weight/day.

III. Methods

The present disclosure provides a method of treating a cancer or a tumor in a subject. The method includes administering a composition that comprises a therapeutically effective amount of 2,4-disulfonyl a-phenyl tert-butyl nitrone (2,4-ds-PBN) to decrease a growth of the cancer or the tumor.

In certain embodiments, administering the effective dose of the composition can treat or prevent the occurrence, recurrence, spread, growth, metastasis, or vascularization of the target tumor or the target cancer. In non-limiting embodiments, the growth of the tumor or cancer can be reduced by at least about 1%, at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65% or at least about 70%.

In non-limiting embodiments, administering the effective dose of the composition can decrease the target tumor and/or cancer's area, a volume, or a combination thereof. In non-limiting embodiments, the tumor and/or cancer volume can be reduced by at least about 1%, at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, or at least about 70%. For example, the non-small cell lung carcinoma's volume can decrease by about 70% after delivering the pharmaceutical composition to the target cancer and/or the target tumor.

In certain embodiments, the method can further include assessing the efficacy of the composition by measuring the volume of cancer or the tumor. For example, the largest diameter (a) and the smallest diameter (b) of each tumor can be measured for estimation of the tumor and/or cancer area or volume at least 1, 2, 3, 4, 8, 12, 18, or 24 times a week, or more.

In certain embodiments, administering the effective dose of the composition can increase apoptosis or necrosis of target tumor and/or cancer cells. For example, a grade of the target tumor and/or cancer's apoptotic or necrosis activity can increase. The grade can be determined by the number of positive cells per histogram area of the target tumor and/or the target cancer (e.g., about 1 mm²). The apoptotic or necrosis grade can be determined by calculating the average number of positive cells and graded into the corresponding severity (e.g., minimal, slight, moderate, marked, and severe). In non-limiting embodiments, a total number or a mean value of the predetermined area of the target cancer and/or the target tumor can decrease after administrating the disclosed composition. For example, the mean value of counting of apoptotic cells can increase by at least about 15%, at least about 20%, at least about 30%, at least about 40%, at least about 50%, at least about 60%, at least about 70%, at least about 80%, at least about 90%, at least about 100%, at least about 110%, at least about 120%, or at least about 130%. In some embodiments, administering the effective dose of the composition can increase apoptosis of target tumor and/or cancer cells can increase the mean value of counting of apoptotic cells by about 130%.

In certain embodiments, the method can further include assessing the efficacy of the composition by counting apoptotic cancer cells or apoptosis tumor cells. For example, the target tumor or cancer can be obtained from a patient and be prepared for hematoxylin & eosin (H&E) staining and TUNEL staining. Necrosis of the obtained cancers/tumors can be evaluated by H&E staining, and apoptosis of the obtained cancers/tumors can be evaluated by the number of TUNEL staining positive cells per predetermined area (e.g., 1 mm²). In some embodiments, the apoptosis of the tumor and/or cancer can be determined by calculating the average number of positive cells and graded into the corresponding severity (e.g., minimal, slight, moderate, marked, and severe).

In certain embodiments, the target cancer can be non-small cell lung carcinoma and solid cancers (e.g., gastric cancer, colon cancer, breast cancer, and the like).

Dosage Regimens

For example and not limitation, the administration of the disclosed subject matter can be oral administration, systemic administration, intravenous administration, or local injection to target tissues. An example of oral administration can include delivering the composition containing 2,4-ds-PBN or pharmaceutically acceptable salts to decrease the growth of cancer or the tumor in the form of tablets, pills, capsules, liquids, gels, syrups, slurries, suspensions, and the like to a subject through the mouth of the subject. An example of systemic administration includes intravenous, intraperitoneal, intramuscular, or subcutaneous injections, inhalation (orally or nasally), transdermal, suppository, or enema administration of a composition containing 2,4-ds-PBN or pharmaceutically acceptable salts to decrease the growth of cancer or the tumor. In some embodiments, the pharmaceutical composition can be in the form of a solution for systemic administration, intravenous administration, or local injection to the target cancer or tumor.

In certain embodiments, the following dosage regimens can be used to treat a cancer or a tumor in a subject. In non-limiting embodiments, the composition for oral administration can include the 2,4-ds-PBN or pharmaceutically acceptable salts thereof at least about 5 mg/kg body weight/day, at least about 10 mg/kg body weight/day, at least about 20 mg/kg body weight/day, at least about 50 mg/kg body weight/day, at least about 100 mg/kg body weight/day, at least about 200 mg/kg body weight/day, at least about 300 mg/kg body weight/day, at least about 400 mg/kg body weight/day, at least about 500 mg/kg body weight/day, at least about 600 mg/kg body weight/day, at least about 700 mg/kg body weight/day, at least about 800 mg/kg body weight/day, at least about 900 mg/kg body weight/day, or at least about 1000 mg/kg body weight/day. In non-limiting embodiments, the effective dose for oral administration can be from about 5 mg/kg body weight/day to about 1,000 mg/kg body weight/day, from about 10 mg/kg body weight/day to about 1,000 mg/kg body weight/day, from about 10 mg/kg body weight/day to about 400 mg/kg body weight/day, from about 10 mg/kg body weight/day to about 300 mg/kg body weight/day, from about 10 mg/kg body weight/day to about 200 mg/kg body weight/day, from about 10 mg/kg body weight/day to about 100 mg/kg body weight/day, from about 20 mg/kg body weight/day to about 1,000 mg/kg body weight/day, from about 50 mg/kg body weight/day to about 1,000 mg/kg body weight/day, from about 50 mg/kg body weight/day to about 600 mg/kg body weight/day, from about 50 mg/kg body weight/day to about 400 mg/kg body weight/day, from about 50 mg/kg body weight/day to about 200 mg/kg body weight/day, from about 60 mg/kg body weight/day to about 1,000 mg/kg body weight/day, from about 70 mg/kg body weight/day to about 1,000 mg/kg body weight/day, from about 80 mg/kg body weight/day to about 1,000 mg/kg body weight/day, from about 90 mg/kg body weight/day to about 1,000 mg/kg body weight/day, from about 100 mg/kg body weight/day to about 1,000 mg/kg body weight/day, from about 200 mg/kg body weight/day to about 1,000 mg/kg body weight/day, from about 300 mg/kg body weight/day to about 1,000 mg/kg body weight/day, from about 400 mg/kg body weight/day to about 1,000 mg/kg body weight/day, from about 500 mg/kg body weight/day to about 1,000 mg/kg body weight/day, from about 600 mg/kg body weight/day to about 1,000 mg/kg body weight/day, from about 700 mg/kg body weight/day to about 1,000 mg/kg body weight/day, from about 800 mg/kg body weight/day to about 1,000 mg/kg body weight/day, from about 5 mg/kg body weight/day to about 900 mg/kg body weight/day, from about 5 mg/kg body weight/day to about 800 mg/kg body weight/day, from about 5 mg/kg body weight/day to about 700 mg/kg body weight/day, from about 5 mg/kg body weight/day to about 600 mg/kg body weight/day, from about 5 mg/kg body weight/day to about 500 mg/kg body weight/day, from about 5 mg/kg body weight/day to about 400 mg/kg body weight/day, from about 5 mg/kg body weight/day to about 300 mg/kg body weight/day, from about 5 mg/kg body weight/day to about 200 mg/kg body weight/day, or from about 5 mg/kg body weight/day to about 100 mg/kg body weight/day.

In certain embodiments, the composition can be administered to a subject by orally administering the disclosed dose of the composition to the subject, 1 to 4 times daily. For example, the composition can be applied 1, 2, 3, or 4 times a day or more. In certain embodiments, the composition can be applied by administering the disclosed dose of the composition to the subject, once daily, twice daily, three times daily, or four times daily. For example and not limitation, the composition can be applied by administering the disclosed dose of the composition to the target tissue three times daily, including, for example, in the morning, noon, and evening. In non-limiting embodiments, the disclosed composition can be administered in appropriate amounts divided into several portions for a specific period of time.

In certain embodiments, the composition for the systemic administration, intravenous administration, or local injection can include the 2,4-ds-PBN or pharmaceutically acceptable salts thereof at least about 10 mg/kg body weight/day, at least about 20 mg/kg body weight/day, at least about 30 mg/kg body weight/day, at least about 40 mg/kg body weight/day, at least about 50 mg/kg body weight/day, at least about 60 mg/kg body weight/day, at least about 70 mg/kg body weight/day, at least about 80 mg/kg body weight/day, at least about 90 mg/kg body weight/day, at least about 100 mg/kg body weight/day, at least about 200 mg/kg body weight/day, at least about 300 mg/kg body weight/day, at least about 400 mg/kg body weight/day, or at least about 500 mg/kg body weight/day. In non-limiting embodiments, the effective dose for the systemic administration, intravenous administration, or local injection can be from about 10 mg/kg body weight/day to about 500 mg/kg body weight/day, from about 20 mg/kg body weight/day to about 500 mg/kg body weight/day, from about 30 mg/kg body weight/day to about 500 mg/kg body weight/day, from about 40 mg/kg body weight/day to about 500 mg/kg body weight/day, from about 50 mg/kg body weight/day to about 500 mg/kg body weight/day, from about 60 mg/kg body weight/day to about 500 mg/kg body weight/day, from about 70 mg/kg body weight/day to about 500 mg/kg body weight/day, from about 80 mg/kg body weight/day to about 500 mg/kg body weight/day, from about 90 mg/kg body weight/day to about 500 mg/kg body weight/day, from about 100 mg/kg body weight/day to about 500 mg/kg body weight/day, from about 200 mg/kg body weight/day to about 500 mg/kg body weight/day, from about 300 mg/kg body weight/day to about 500 mg/kg body weight/day, from about 20 mg/kg body weight/day to about 500 mg/kg body weight/day, from about 20 mg/kg body weight/day to about 400 mg/kg body weight/day, from about 20 mg/kg body weight/day to about 300 mg/kg body weight/day, from about 20 mg/kg body weight/day to about 200 mg/kg body weight/day, from about 20 mg/kg body weight/day to about 100 mg/kg body weight/day, from about 60 mg/kg body weight/day to about 400 mg/kg body weight/day, from about 60 mg/kg body weight/day to about 300 mg/kg body weight/day, or from about 60 mg/kg body weight/day to about 200 mg/kg body weight/day.

In certain embodiments, the composition for the systemic administration, intravenous administration, or local injection can be prepared in the liquid solutions or suspensions in an aqueous physiological buffer.

The composition for the systemic administration, intravenous administration, or local injection can be administered to the target cancer and/or the target tumor by delivering the composition prepared for systemic, intravenous, or local application to the target region 1, 2, 3, 4, 5, 6, or 7 times a week. In non-limiting embodiments, the pharmaceutical composition can be delivered once a week or two times a week, or three times a week, or four times a week, or 5 times a week.

In certain embodiments, the dosage administered can vary depending upon known factors, such as the route of administration, age, health, and weight of the recipient, nature and extent of symptoms, kind of concurrent treatment, frequency of treatment, and the effect desired. In non-limiting embodiments, the disclosed dosage regimes can be adjusted over time according to the individual need and the professional judgment of the person administering or supervising the administration of the composition. For example, the dosage of the composition can be increased if the lower dose does not provide sufficient activity in the treatment of the condition described herein (e.g., non-small cell lung carcinoma). Alternatively, the dosage of the composition can be decreased if the disease (e.g., non-small cell lung carcinoma) is reduced, no longer detectable, or eliminated.

In certain embodiments, the disclosed composition can be administered to the subject in a single dose or divided doses.

In certain embodiments, the duration of the disclosed treatment can be between about one day to about five years. In certain embodiments, the duration of the disclosed treatment can be about at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 2 years at least about 3 years, at least about 4 years, or at least about 5 years. In certain embodiments, the composition can be administered until the cancers/tumors are no longer detectable.

Co-Administration

In certain embodiments, the composition disclosed herein can be used alone or in combination with one or more therapies. For example, but not by way of limitation, methods of the present disclosure can include administering the disclosed compositions and one or more anti-tumor or anti-cancer therapies. “In combination with,” as used herein, means that the disclosed composition and the one or more therapies can be administered to a subject as part of a treatment regimen or plan. In certain embodiments, being used in combination does not require that the composition and one or more agents for the additional therapies are physically combined prior to administration, administered by the same route or that they be administered over the same time frame. In certain embodiments, the agent for additional therapies can be administered before the disclosed composition. In certain embodiments, the agent for additional therapies can be administered after the disclosed composition. In certain embodiments, the agent for additional therapies can be administered simultaneously with the disclosed composition. Non-limiting exemplary additional therapies can include, but are not limited to, the anti-tumor treatment, anti-cancer treatment, chemotherapy, targeted therapy, immunotherapy, radiation, radiofrequency ablation, surgery, therapy using a tumor treating fields (TTFields) device, or combinations thereof. In non-limiting embodiments, the agent for the additional therapies can include cisplatin, cyclophosphamide, doxorubicin, vincristine, topotecan, pemetrexed, gefitinib, erlotinib, dacomitinib, osimertinib, nivolumab, pembrolizumab, atezolizumab, durvalumab, chemotherapeutic agents, radiotherapeutic agents, cytokines, anti-angiogenic agents, tyrosine kinase inhibitors (TKI), apoptosis-inducing agents, anti-cancer antibodies, a targeted drug, and/or agents which promote the activity of the immune system, including but not limited to cytokines such as but not limited to interleukin 2 (IL-2), interferon, an anti-CTLA4 antibody, an anti-PD-1 antibody and/or an anti-PD-L1 antibody, immune checkpoint inhibitors, immune cell therapy agents, therapeutic antibodies, anticancer vaccines, or combinations thereof. In certain embodiments, the anti-cancer agent can be a taxane, a platinum-based agent, an anthracycline, an anthraquinone, an alkylating agent, a HER2 targeting therapy, vinorelbine, a nucleoside analog, ixabepilone, eribulin, cytarabine, a hormonal therapy, methotrexate, capecitabine, lapatinib, 5-FU, etoposide or any combination thereof.

In certain embodiments, administering the disclosed compositions in combination with one or more additional therapies can induce combinatorial or synergistic effects for decreasing or preventing target tumor and/or cancer growth. For example, but not by way of limitation, synergistic decreases in the growth of area and volume from the target cancer and/or the target tumor can occur when combinations of the disclosed composition and additional-therapies are delivered to a subject. In non-limiting embodiments, the disclosed composition can suppress the target tumor and/or cancer growth and increase the sensitivity of the additional therapies for the target cancer and/or tumor treatment.

In non-limiting embodiments, the disclosed method for treating a cancer or a tumor in a subject in need thereof can include administering a therapeutically effective amount of 2,4-ds-PBN or pharmaceutically acceptable salts thereof, measuring a volume, an area, or a combination thereof of the cancer or the tumor, counting apoptotic cancer cells or apoptotic tumor cells, administering a therapeutically effective amount of an anti-cancer agent or an anti-tumor agent to the subject, and administering an additional therapy, wherein the therapeutically effective amount can decrease a growth of the cancer or the tumor, wherein the cancer can be a non-small cell lung carcinoma, wherein the therapeutically effective amount of the 2,4-ds-PBN or pharmaceutically acceptable salts thereof can be from about 20 mg/kg body weight/day to about 1,200 mg/kg body weight/day, wherein the therapeutically effective amount of the 2,4-ds-PBN or pharmaceutically acceptable salts thereof can be delivered to the subject via an intravenous injection or an intra-arterial injection or alternatively the therapeutically effective amount of the 2,4-ds-PBN or pharmaceutically acceptable salts thereof can be delivered to the subject via an enteral administration or an oral administration, wherein the cancer volume or the tumor volume can decrease by at least about 50% after administrating the therapeutically effective amount of the 2,4-ds-PBN or pharmaceutically acceptable salts thereof, wherein a growth of the cancer or the tumor can decrease by at least about 40% after administrating the therapeutically effective amount of the 2,4-ds-PBN or pharmaceutically acceptable salts thereof, wherein a number of the apoptotic cancer cells or the apoptotic tumor cells can increase by at least about 15% after administrating the therapeutically effective amount of the 2,4-ds-PBN or pharmaceutically acceptable salts thereof, wherein the therapeutically effective amount of the 2,4-ds-PBN or pharmaceutically acceptable salts thereof can be delivered to the subject at least once a week, wherein the anti-cancer/tumor agent can be selected from the group consisting of a chemotherapeutic agent, an immunotherapeutic agent, a cytokine, an anti-angiogenic agent, a tyrosine kinase inhibitor (TKI), an apoptosis-inducing agent, an anti-cancer antibody, and combinations thereof, wherein the additional therapy can be selected from the group consisting of an anti-tumor therapy, an anti-cancer therapy, a chemotherapy, a targeted therapy, an immunotherapy, a radiation therapy, a radiofrequency ablation therapy, surgery, a therapy using a tumor treating fields (TTFields) device, or combinations thereof.

### Examples

The presently disclosed subject matter will be better understood by reference to the following Examples, which are provided as exemplary of the presently disclosed subject matter, and not by way of limitation

### EXAMPLE: Anti-Carcinogenic Effects of OKN-007 on Human Lung Cancer Cell Line A549 Xenografted in Nude Mice

The anti-cancer efficacy of OKN-007 was assessed after oral administration in nude mice carrying human A549 lung cancer xenografts implanted subcutaneously in the flank.

Materials and Methods:

Test Substance was OKN-007 that refers to 2,4-disulfonyl a-phenyl tert-butyl nitrone (2,4-ds-PBN, Disodium 4-[(Z)-[tert-butyl(oxido)azaniumylidene]methyl]benzene-1,3-disulfonate) and has the molecular formula: C₁₁H₁₃NO₇S₂.Na₂.

The positive substance was Cis-Diammineplatinum (II) dichloride (Cisplatin), which has the molecular formula: Pt (NH₃)₂Cl₂.

Saline was used as a negative control.

Preparation of test substance: The required amount of the test substance was placed in a tube. A small amount of vehicles was added and mixed. The vehicle was gradually added to yield the desired concentrations (e.g., 15, 30, and 60 mg/mL).

Preparation of Positive control: The required amount of the positive control substance was placed in a tube. A small amount of saline was added and mixed. Saline was gradually added to yield the desired concentration (e.g., 0.2 mg/mL).

Human lung cancer cell line, A549 was used.

For Culture medium: Fetal bovine serum (FBS), Penicillin-Streptomycin (10,000 units/mL of penicillin and 10,000 μg/mL of streptomycin), and RPMI1640 were mixed per 100 mL as showing in Table 1.

TABLE 1
Culture medium.

| Name | Amount (mL) |
|---|---|
| FBS (Lot No.: 1982135, Gibco, U.S.A) | 10 |
| Penicillin-Streptomycin (Lot No.: 2019315, Gibco, U.S.A.) | 1 |
| RPMI1640 (0000704247, Lonza, U.S.A.) | 89 |
| Total volume | 100 |

Preparation of cell culture: The frozen cells were thawed in a water bath. Cells were placed in a tube containing the medium and centrifuged (1,000 rpm, 5 minutes) to discard the supernatant. After re-suspending with the culture medium, cells were placed in a cell culture flask and incubated in a 5% CO₂ incubator at 37° C. Cells were sub-cultured at a ratio of 1:5 when grown with confluency of 70-80% in the culture container. Cells cultured on the day of cell line transplantation were centrifuged (1,000 rpm, 5 minutes) to discard the supernatant and to make cell suspension with D-PB S. Then, the cell suspension was mixed with Matrigel at a ratio of 1:1 (5×10⁷ cells/mL).

BALB/c Nude mouse, CAnN.Cg-Foxnlnu/CrlOri, SPF was used. Sex, number, age, and body weight range of animals at receipt: The mice (65 mice) were male, 5 weeks old, and had about 14.5-19.6 g weight at the receipt.

Sex, number, age, and body weight range of animals at the start of administration: The mice (50 mice) were male, 7 weeks old, and had about 18.4-24.0 g weight at the start of administration.

Cell transplantation: After the quarantine-acclimation period, the body weight measurement and cell transplantation in healthy animals were performed. All animals were observed for clinical signs every week. Prepared cell (1×10⁷ cells/0.2 mL) suspension was injected subcutaneously into the right flank with disposable syringes. Animals were observed for clinical signs once daily during the transplantation and engraftment period.

Group assignment: After transplanting cells, each tumor size was monitored for 65 animals. When tumor volume reached within 87-112 mm³, 50 animals were assigned to the respective groups with an average tumor volume of about 99 mm³. Based on the tumor volume, animals were selected and randomly distributed into 5 groups (10 animals/group).

Dosing Route: Oral administration. The route of administration was oral because the clinical application of the test substance is oral.

Method and frequency of administration: For negative control and test substance, oral administration was performed once a day for 4 weeks for a total of 28 times using a disposable syringe. For positive control, intraperitoneal administration was performed once a week for 4 weeks for a total of 4 times using a disposable syringe.

TABLE II
Group designation.

| | | | Dose | Dose | Number of |
|---|---|---|---|---|---|
| | | | (mg/ | volume | animals |
| | Group | Route | kg) | (mL/kg) | (Animal No.) |
| G1 | Negative control | P.O. | 0 | 10 | 10 (1101-1110) |
| G2 | Test substance 1 | P.O. | 150 | 10 | 10 (1201-1210) |
| G3 | Test substance 2 | P.O. | 300 | 10 | 10 (1301-1310) |
| G4 | Test substance 3 | P.O. | 600 | 10 | 10 (1401-1410) |
| G5 | Positive control | I.P. | 2 | 10 | 10 (1501-1510) |

Dosing Route: The dose levels of the test substance were selected at 150, 300, and 600 mg/kg. The dose level of the positive substance was selected at 2 mg/kg. The dose volume was selected at 10 mL/kg for all groups, and the individual dose was calculated based on the bodyweight of animals just prior to dosing.

Measurement of tumor volume: The largest diameter (a) and the smallest diameter (b) of each tumor were measured using calipers twice a week during the observation period. The tumor volume (TV) was calculated twice a week according to the following formula: TV (mm³)=(a (mm)×b² (mm²))/2. The volume of a tumor before administration to each animal was set to the value measured at the time of group assignment.

Tumor removal and measurement of body weight: After the observation period, animals were anesthetized by inhalation of isoflurane. After the tumor was extracted, the weight of the removed tumor was recorded. The tumor growth inhibition rate (IR) was calculated using the following formula: IR (%)=(1−T/C)×100, where T is a mean tumor weight in the test substance group, and positive substance group, and C is a mean tumor weight in the negative control group.

Histopathological test: The extracted tumors were fixed in 10% neutral buffered formalin solution (10% neutral buffered formalin). After sectioning the tumors to a certain thickness (about 3 mm), those were subjected to the general preparation procedure, embedded in paraffin, cut into 4 to 5 μm size, and tissue sections were stained with Hematoxylin and Eosin (H&E) and TUNEL. IHC (Immunohistochemistry) slides, cut into 4 to 5 μm size, were produced and sent to the sponsor (Negative Control Group (G1), Test substance 2 (G3, Animal ID Nos.: 1301, 1302, 1303, 1307, 1310), Test substance 3 (G4, Animal ID Nos.: 1401, 1402, 1404, 1408, 1409)).

Determination of Hematoxylin & Eosin (H&E) staining results: Necrosis of tumors was assessed by grading (±, +, ++, +++, ++++) the extent of necrosis in the whole section of the tumor. Necrosis was evaluated by morphological changes such as collapse or disappearance of the nucleus and eosinophilic change of cytoplasm. The extent of tumor necrosis was determined by the percentage of the following necrosis area.

TABLE 3
Necrosis grading.

| Severity | Rate (%) |
|---|---|
| Minimal (±) | 0~15 |
| Slight (+) | 15~45 |
| Moderate (++) | 45~70 |
| Marked (+++) | 70~85 |
| Severe (++++) | 85~ |

Determination of TUNEL staining results: After histological examination of the entire cross-section of the tumor for grading (±, +, ++, +++, ++++) of apoptosis, the nucleated brown stained cells were read as benign cells, and the number of benign cells was counted. The apoptosis of the tumor was determined by the number of positive cells per histogram area (1 mm²). The apoptosis of the tumor was determined by calculating the average number of positive cells and graded into the corresponding severity. The number of positive cells in which apoptosis occurred was determined by counting the number of positive cells per unit area (1 mm²).

TABLE 4
Apoptosis grading

| Severity | Apoptosis No. |
|---|---|
| Minimal (±) | 5~10 |
| Slight (+) | 11~20 |
| Moderate (++) | 21~30 |
| Marked (+++) | 31~40 |
| Severe (++++) | 41~ |

Statistical Analysis: Statistical analysis was conducted using a statistical program (Version 9.3, SAS Institute Inc., U.S.A.) for the data, including body weights, tumor volume, tumor weight, and histopathological test. Bartlett's test was employed on the homogeneity of variance (significance level: 0.05). One-way analysis of variance (ANOVA) was employed on homogeneous data. Then if significant, Dunnett's t-test was applied for multiple comparisons (significance levels: 0.05 and 0.01, one-tailed). Kruskal-Wallis test was employed on overridden homogeneous data. Then if significant, Steel's test was applied for multiple comparisons (significance levels: 0.05 and 0.01, one-tailed).

Results

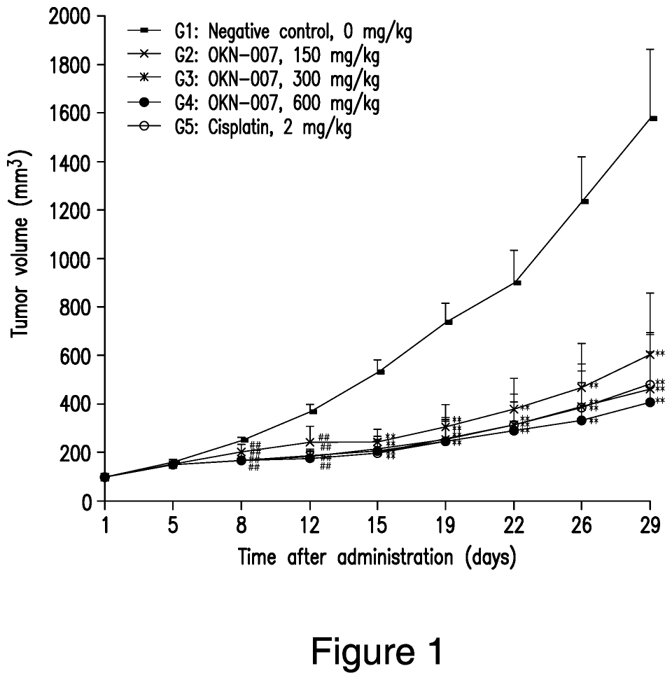

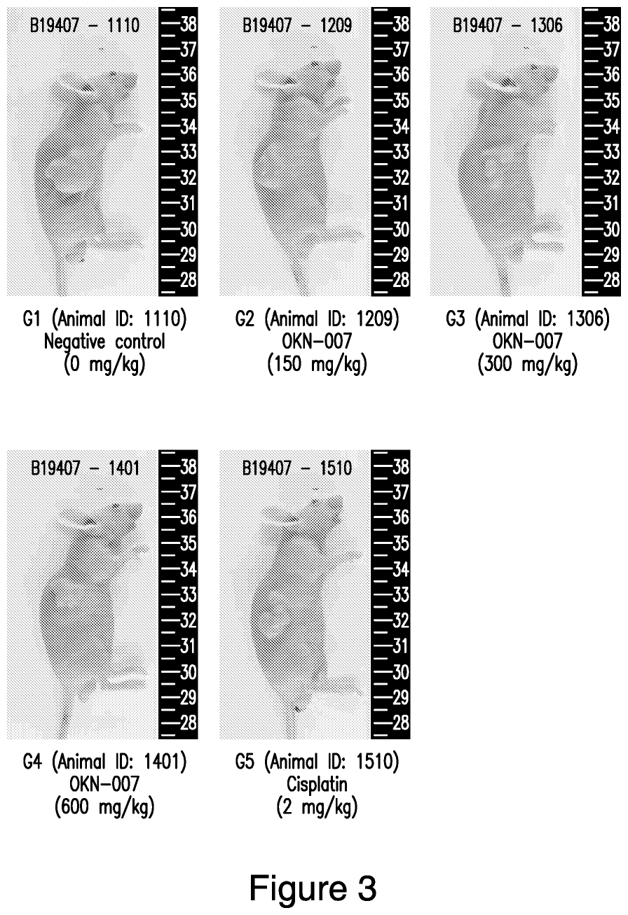

Change of tumor volume (FIGS. 1, 3, 4, 5, 6, and Table 5): In the negative control group (G1), the mean tumor volume ranged from 99 to 1576 mm³ before and 29 days after the administration of the control, with a tendency to increase over time (FIGS. 1, 3, and 4).

TABLE 5
Mean tumor volume during the observation period.

| Group/ | | Tumor volume (mm³) | | | | | | | | |
|---|---|---|---|---|---|---|---|---|---|---|
| Dose | | Time after administration (days) | | | | | | | | |
| (mg/kg) | | 1 | 5 | 8 | 12 | 15 | 19 | 22 | 26 | 29 |
| G1 | Mean | 99 | 160 | 248 | 366 | 531 | 735 | 897 | 1235 | 1576 |
| 0 | S.D. | 8 | 13 | 14 | 33 | 52 | 80 | 138 | 185 | 287 |
| | N | 10 | 10 | 10 | 10 | 10 | 10 | 10 | 10 | 10 |
| G2 | Mean | 99 | 152 | 200 | 240 | 243 | 306 | 376 | 466 | 603 |
| 150 | S.D. | 8 | 14 | 35 | 66 | 54 | 92 | 130 | 183 | 254 |
| | N | 10 | 10 | 10 | 10 | 10 | 10 | 10 | 10 | 10 |
| | | | | ^{##} | ^{##} | ^{**} | ^{**} | ^{**} | ^{**} | ^{**} |
| G3 | Mean | 99 | 146 | 172 | 182 | 212 | 254 | 310 | 390 | 460 |
| 300 | S.D. | 8 | 16 | 14 | 24 | 54 | 90 | 131 | 177 | 228 |
| | N | 10 | 10 | 10 | 10 | 10 | 10 | 10 | 10 | 10 |
| | | | | ^{##} | ^{##} | ^{**} | ^{**} | ^{**} | ^{**} | ^{**} |
| G4 | Mean | 99 | 149 | 176 | 179 | 209 | 249 | 288 | 331 | 407 |
| 600 | S.D. | 8 | 13 | 13 | 26 | 46 | 86 | 121 | 159 | 210 |
| | N | 10 | 10 | 10 | 10 | 10 | 10 | 10 | 10 | 10 |
| | | | | ^{##} | ^{##} | ^{**} | ^{**} | ^{**} | ^{**} | ^{**} |
| G5 | Mean | 99 | 152 | 171 | 176 | 199 | 250 | 314 | 385 | 481 |
| 2 | S.D. | 8 | 11 | 26 | 37 | 51 | 79 | 127 | 152 | 216 |
| | N | 10 | 10 | 10 | 10 | 10 | 10 | 10 | 10 | 10 |
| | | | | ^{##} | ^{##} | ^{**} | ^{**} | ^{**} | ^{**} | ^{**} |

In Table 5, G1 refers to a negative control group. G2, G3, and G4 refer to OKN-007 treated groups. G5 refers to a cisplatin-treated group. S.D refers to a standard deviation. N refers to the number of animals. ## p<0.01 refers to a significant difference from the negative control group (G1) by Steel's t-test. ** p<0.01 refers to a significant difference from the negative control group (G1) by Dunnett's t-test.

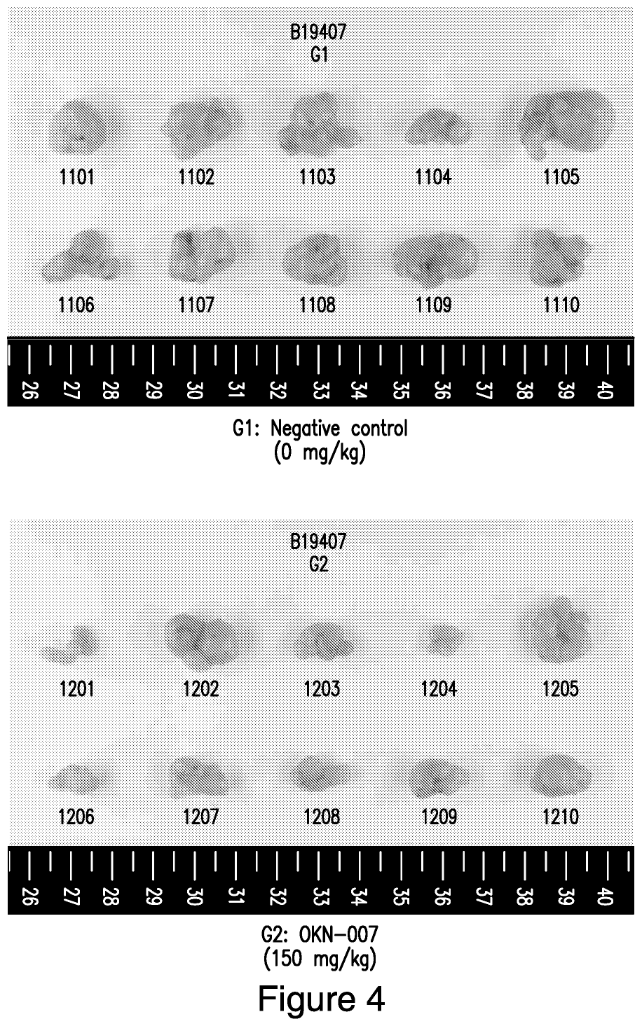

In the 150 mg/kg test substance 1 (OKN-007) group (G2), there was a decrease in mean tumor volume, ranging from 99 to 603 mm³, which reaches statistical significance of difference (p<0.01: 8 to 29 days after the administration) from the negative control group (G1) at a certain measurement point from all dose levels. See FIGS. 1 and 4.

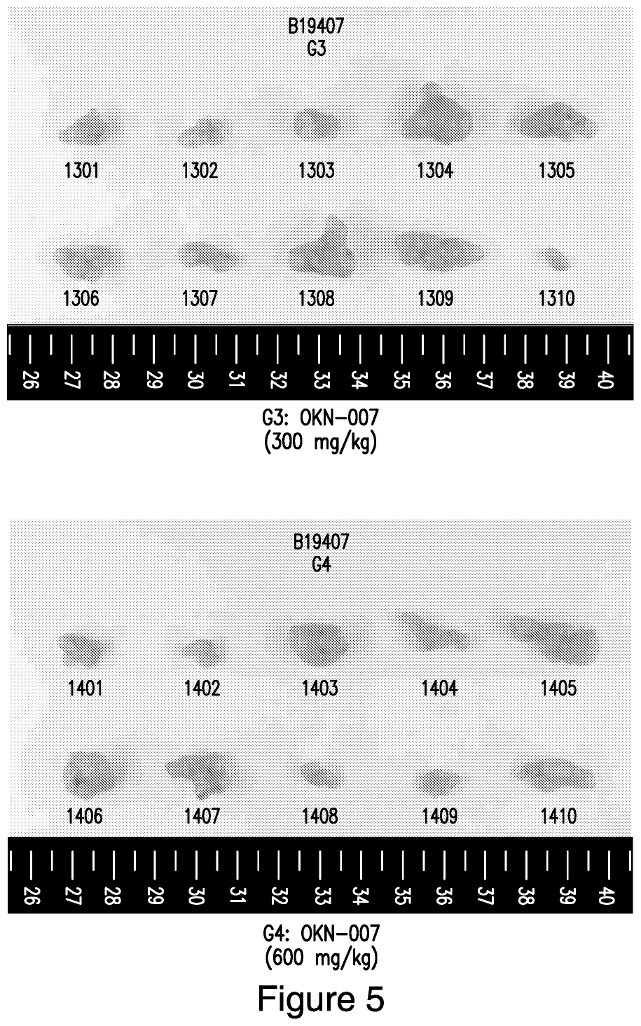

In the 300 mg/kg test substance 2 (OKN-007) group (G3), there was a decrease in mean tumor volume, ranging from 99 to 460 mm³, which reaches statistical significance of difference (p<0.01: 8 to 29 days after the administration) from the negative control group (G1) at a certain measurement point from all dose levels. See FIGS. 1 and 5.

In the 600 mg/kg test substance 3 (OKN-007) group (G4), there was a decrease in mean tumor volume, ranging from 99 to 407 mm³, which reaches statistical significance of difference (p<0.01: 8 to 29 days after the administration) from the negative control group (G1) at a certain measurement point from all dose levels. See FIGS. 1 and 5.

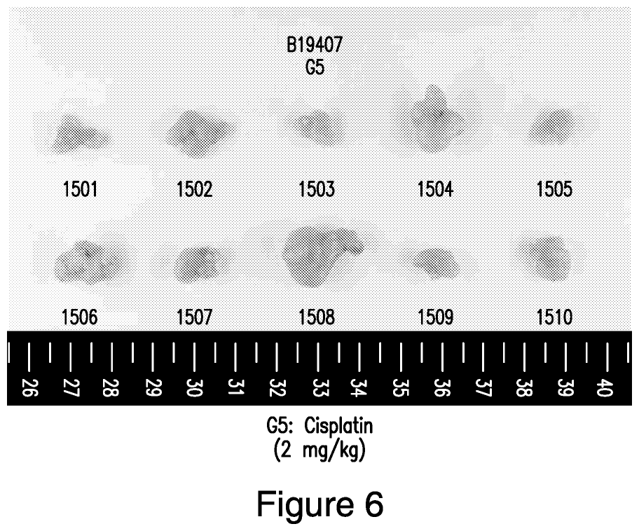

In the 2 mg/kg positive control (Cisplatin) group (G5), there was a decrease in mean tumor volume, ranging from 99 to 481 mm³, which reaches statistical significance of difference (p<0.01: 8 to 29 days after the administration) from the negative control group (G1) at a certain measurement point from all dose levels. See FIGS. 1 and 6.

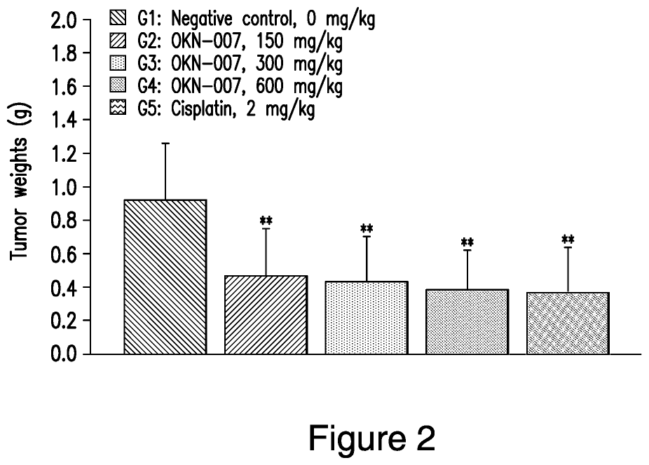

Tumor weights and growth inhibition rate (FIG. 2, 3, 4, 5, 6, and Table 6): The tumors were extirpated and weighted at day 29. In the negative control group (G1), the average tumor weight was 0.92 g (tumor growth inhibition rate (IR): 0.0%).

TABLE 6
Mean data of tumor weights and tumor
growth inhibition rate.

| Group/ | | Tumor | IR |
|---|---|---|---|
| Dose (mg/kg) | | weights (g) | (%) |
| G1 | Mean | 0.92 | 0.0 |
| 0 | S.D. | 0.34 | |
| | N | 10 | |
| G2 | Mean | 0.47 | 48.9 |
| 150 | S.D. | 0.28 | |
| | N | 10 | |
| | | ** | |
| G3 | Mean | 0.43 | 53.3 |
| 300 | S.D. | 0.27 | |
| | N | 10 | |
| | | ** | |
| G4 | Mean | 0.38 | 58.7 |
| 600 | S.D. | 0.24 | |
| | N | 10 | |
| | | ** | |
| G5 | Mean | 0.37 | 59.8 |
| 2 | S.D. | 0.27 | |
| | N | 10 | |
| | | ** | |

In Table 6, G1 refers to a negative control group. G2, G3, and G4 refer to OKN-007 treated groups. G5 refers to a cisplatin-treated group. S.D refers to a standard deviation. IR refers to the following equation: (Tumor growth inhibition rate, %)=(1— T/C)×100, where T is a mean tumor weight of the test and positive substance group, and C is a mean tumor weight of the negative control group. ** p<0.01 refers to a significant difference from the negative control group (G1) by Dunnett's t-test. See FIG. 2.

In the 150 mg/kg test substance 1 (OKN-007) group (G2), the mean value of tumor weights was 0.47 g (tumor growth inhibition rate (IR): 48.9%). There was the statistical significance of the difference between the negative control and group 2. See FIG. 2.

In the 300 mg/kg test substance 2 (OKN-007) group (G3), the mean value of tumor weights was 0.43 g (tumor growth inhibition rate (IR): 53.3%). There was the statistical significance of the difference between the negative control and group 3. See FIG. 2.

In the 600 mg/kg test substance 3 (OKN-007) group (G4), the mean value of tumor weights was 0.38 g (tumor growth inhibition rate (IR): 58.7%). There was the statistical significance of the difference between the negative control and group 4. See FIG. 2.

In the 2 mg/kg positive control (Cisplatin) group (G5), the mean value of tumor weights was 0.37 g (tumor growth inhibition rate (IR): 59.8%). There was the statistical significance of the difference between the negative control and group 5. See FIG. 2.

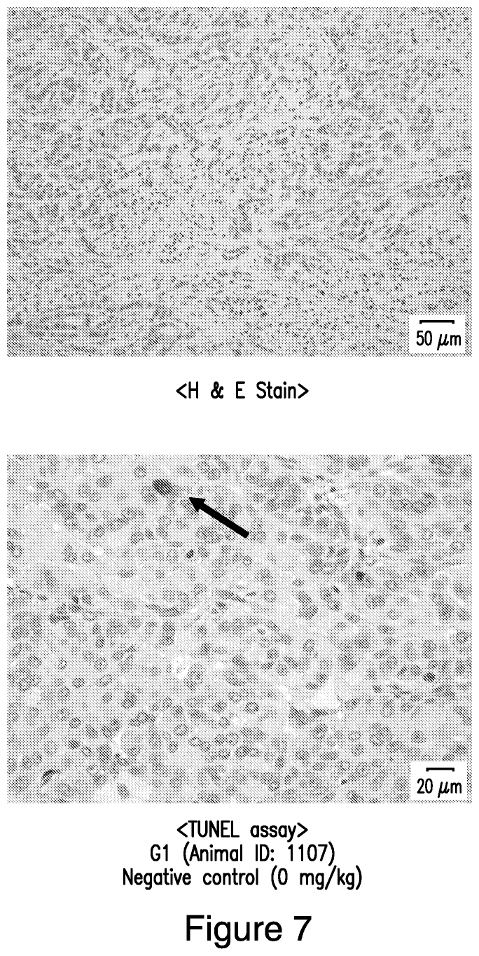

Histopathological test (Table 7, FIGS. 7-11): The extracted fixed tumor was cut to a certain thickness (about 3 mm), then subjected to general tissue treatment, paraffin-embedded to produce blocks, and tissue sections of 4 to 5 μm in size were prepared. Then, Hematoxylin & Eosin (H&E) staining and TUNEL staining were performed.

Necrosis of extracted tumors was evaluated by H&E staining. As a result, the severity of tumor necrosis was mostly observed at the lowest severity in all treatment groups, which from the necrosis grading applies to minimal, slight, and moderate severity. The severity of necrosis between the test substance groups (G2, G3, and G4) and the positive control (Cisplatin) group (G5) does not significantly different compared to that of the negative control group (G1).

Apoptosis was confirmed by the TUNEL assay, and the apoptosis cells were counted accordingly. As a result, apoptosis increased in all administration groups compared to the negative control group (G1). Apoptosis was increased in all administration groups.

TABLE 7
Summary of Histopathological Findings.

| Group/ | | Counting | | | | | | | | | | |
|---|---|---|---|---|---|---|---|---|---|---|---|---|
| (mg/kg) | | No.^{*} | Apoptosis | | | | | Necrosis | | | | |
| Dose | N | (No./mm²⁾ | ± | + | ++ | +++ | ++++ | ± | + | ++ | +++ | ++++ |
| G1/0 | 10 | 65.3 ± 27.7 | 9 | 1 | 0 | 0 | 0 | 5 | 4 | 1 | 0 | 0 |
| G2/150 | 10 | 79.6 ± 20.8^{##} | 9 | 1 | 0 | 0 | 0 | 5 | 3 | 2 | 0 | 0 |
| G3/300 | 10 | 91.5 ± 32.2^{##} | 8 | 2 | 0 | 0 | 0 | 7 | 2 | 1 | 0 | 0 |
| G4/600 | 10 | 149.9 ± 59.5^{##} | 3 | 5 | 2 | 0 | 0 | 6 | 2 | 2 | 0 | 0 |
| G5/2 | 10 | 87.5 ± 50.6 | 8 | 2 | 0 | 0 | 0 | 6 | 2 | 2 | 0 | 0 |

In Table 7, G1 refers to a negative control group. G2, G3, and G4 refer to OKN-007 treated groups. G5 refers to a cisplatin-treated group. S.D refers to a standard deviation. N refers to the number of animals. ## p<0.01 refers to a significant difference from the negative control group (G1) by Steel's t-test. * refers to group means±S.D. refers to a minimal grade. + refers to a mild grade, ++ refers to a moderate grade, +++ refers to a marked grade, ++++ refers to a severe grade. Necrosis is determined by H&E Stain analyses. Apoptosis is determined by TUNEL assay.

In the negative control group (G1), the mean value of apoptosis counting was 65.3±27.7. See FIG. 7 and Table 7.

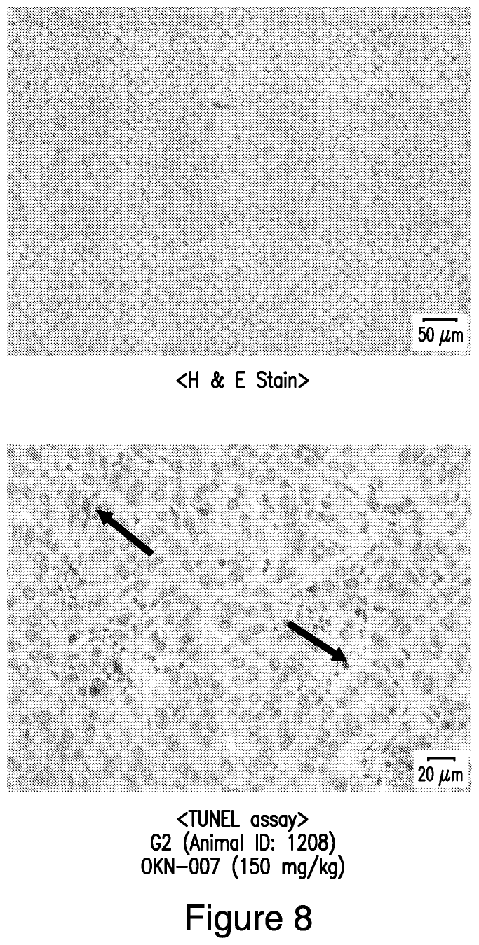

In the 150 mg/kg test substance 1 (OKN-007) group (G2), the mean value of apoptosis counting was 79.6±29.8. There was a statistically significant difference (p<0.01) compared to the negative control group (G1). See FIG. 8 and Table 7.

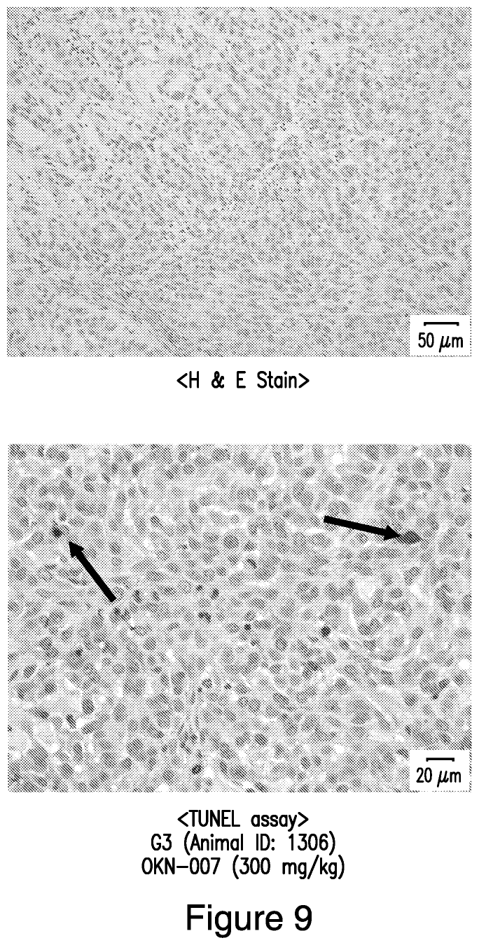

In the 300 mg/kg test substance 2 (OKN-007) group (G3), the mean value of apoptosis counting was 91.5±32.2. There was a statistically significant difference (p<0.01) compared to the negative control group (G1). See FIG. 9 and Table 7.

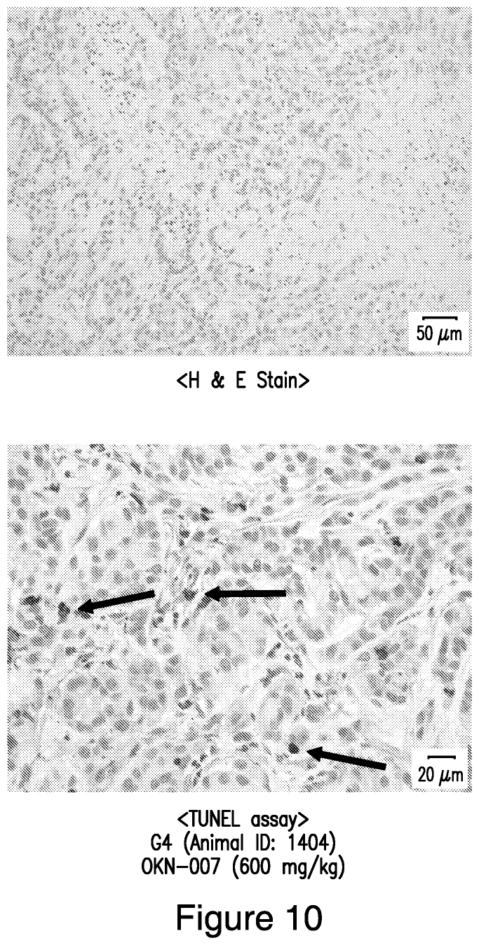

In the 600 mg/kg test substance 3 (OKN-007) group (G4), the mean value of apoptosis counting was 149.9±59.5. There was a statistically significant difference (p<0.01) compared to the negative control group (G1). See FIG. 10 and Table 7.

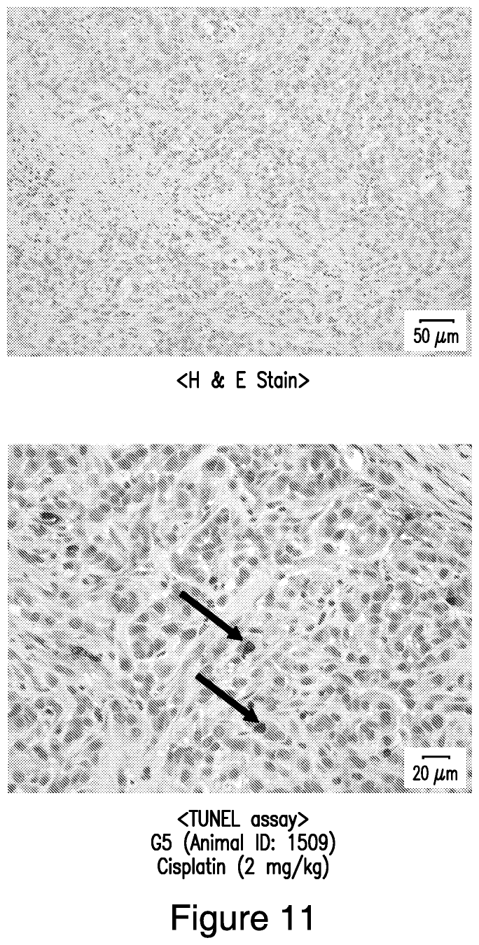

In the 2 mg/kg positive control (Cisplatin) group (G5), the mean value of apoptosis counting was 87.5±50.6. There was no statistically significant difference compared to the negative control group (G1). See FIG. 11 and Table 7.

As a result, mice that received 600 mg/kg test substance per oral gavage (G4) had the highest apoptosis counting among all groups, then 300 mg/kg test substance group (G3), 2 mg/kg positive control (Cisplatin) group (G5) and 150 mg/kg test substance group (G2) compared with the negative control group (G1).

### DISCUSSION

The purpose of this study was to evaluate in vivo anti-cancer efficacy of the test substance OKN-007 after its oral administration in nude mice carrying human A549 lung cancer xenografts implanted subcutaneously in the flank.

The human tumor xenograft system carried out in this study provides a useful model for experimental cancer therapy studies as nude mice are known for their weak immune rejection due to the deficiency of T-lymphocytes.

Tumor inhibition efficacy was evaluated by measuring the tumor volume and weight. When the values of tumor volume and weight were reduced compared to the negative control group and such results were considered statistically significant, the test substance was regarded as effective for anti-tumor therapy.

In tumor volume, the smallest to largest tumor volume was 600 mg/kg test substance group (G4), then 300 mg/kg test substance group (G3), 2 mg/kg positive control (Cisplatin) group (G5), and 150 mg/kg test substance group (G2) compared to the negative control group (G1). The tumor volume is correlated with the tumor area. For example, similar to the data related to decreased tumor volume, the tumor area and/or growth of tumor area decreases after the OKN-007 treatment.

In tumor weight, 2 mg/kg positive control (Cisplatin) group (G5) showed the lowest. This suggests that the positive control has tumor growth inhibitory effect on tumor growth. The next lowest tumor weight was 600 mg/kg test substance group (G4), 300 mg/kg test substance group (G3), 150 mg/kg test substance group (G2), and the negative control group being the highest.

Regarding tumor growth inhibition rate, the highest to lowest were 2 mg/kg positive control (Cisplatin) group (G5) resulting 59.8% inhibition, then 600 mg/kg test substance group (G4) (58.7% inhibition), 300 mg/kg test substance group (G3) (53.5% inhibition) and 150 mg/kg test substance group (G2) (48.9% inhibition) when compared to the negative control (0.0% inhibition).

Moreover, the statistical analysis and the gravimetric measurement data of the extracted tumors from all study groups were considered statistically significant.

Apoptosis was evaluated with TUNEL assay, and the result of the assay was an increase in apoptosis in all administration groups compared to the negative control. 600 mg/kg test substance group (G4) was showing the highest level of apoptotic activity among all groups, then 300 mg/kg test substance group (G3), 2 mg/kg positive control (Cisplatin) group (G5), and 150 mg/kg test substance group (G2) compared to the negative control. All of the test groups except the positive control were considered statistically significant.

It was determined that the tumor growth inhibitory effect of the test substance was dose-dependent, and the test substance was considered to have a tumor growth inhibitory effect.

In conclusion, the dose-dependent inhibitory effect of the test substance (OKN-007) on tumor growth was observed in human lung cancer A549 xenografts in nude mice. A clinically relevant amount of OKN-007 can be used for human applications to induce the clinically relevant anti-caner and/or anti-tumor effects. For example, OKN-007 at a concentration range from about 10 mg/kg body weight/day to about 500 mg/kg body weight/day can be used for systemic, intravenous, or local administration to a human subject. OKN-007 at a concentration range from about 5 mg/kg body weight/day to about 1,000 mg/kg body weight/day can be used for oral or enteral administration to a human subject.

### REFERENCES

Guanjai Dai et al., Myricanol Induces Apoptotic Cell Death and Anti-Tumor Activity in Non-Small Cell Lung Carcinoma in Vivo, International Journal of Molecular Sciences. 2015 Jan.; 16(2): 2717-2731.
Rhyu J J et al., Dual effects of human adipose tissue-derived mesenchymal stem cells in human lung adenocarcinoma A549 xenografts and colorectal adenocarcinoma HT-29 xenografts in mice, Oncology Reports. 2015 Oct.; 34(4): 1733-1744.
Towner, et al., OKN-007 Increases temozolomide (TMZ) Sensitivity and Suppresses TMZ-Resistant Glioblastoma (GBM) Tumor Growth, Translational Oncology, 2019 Feb.; 12(2): 320-335.
Towner, et al., Regression of glioma tumor growth in F98 and U87 rat glioma models by the Nitrone OKN-007, Neuro Oncology 2013 Jan.; 15(3): 330-340

All patents, patent applications, publications, product descriptions, and protocols cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

Various publications and nucleic acid and amino acid sequence accession numbers are cited herein, the contents and full sequences of which are hereby incorporated by reference herein in their entireties.

1. A method for treating a cancer or a tumor in a subject in need thereof, wherein the method comprises administering a therapeutically effective amount of 2,4-disulfonyl a-phenyl tert-butyl nitrone (2,4-ds-PBN) or pharmaceutically acceptable salts thereof, wherein the therapeutically effective amount decreases a growth of the cancer or the tumor.

2. The method of claim 1, wherein the cancer is a non-small cell lung carcinoma.

3. The method of claim 1, wherein the therapeutically effective amount of the 2,4-ds-PBN or pharmaceutically acceptable salts thereof is from about 5 mg/kg body weight/day to about 1,000 mg/kg body weight/day.

4. The method of claim 1, wherein the therapeutically effective amount of the 2,4-ds-PBN or pharmaceutically acceptable salts thereof is delivered to the subject via an intravenous injection or an intra-arterial injection.

5. The method of claim 1, wherein the therapeutically effective amount of the 2,4-ds-PBN or pharmaceutically acceptable salts thereof is delivered to the subject via an enteral administration or an oral administration.

6. The method of claim 1, further comprising measuring a volume, an area, or a combination thereof of the cancer or the tumor.

7. The method of claim 6, wherein the cancer volume or the tumor volume decreases by at least about 50% after administrating the therapeutically effective amount of the 2,4-ds-PBN or pharmaceutically acceptable salts thereof.

8. The method of claim 6, wherein the growth of the cancer or the tumor decreases by at least about 40% after administrating the therapeutically effective amount of the 2,4-ds-PBN or pharmaceutically acceptable salts thereof.

9. The method of claim 1, further comprising counting apoptotic cancer cells or apoptotic tumor cells.

10. The method of claim 9, wherein a number of the apoptotic cancer cells or the apoptotic tumor cells increases by at least about 15% after administrating the therapeutically effective amount of the 2,4-ds-PBN or pharmaceutically acceptable salts thereof.

11. The method of claim 1, the therapeutically effective amount of the 2,4-ds-PBN or pharmaceutically acceptable salts thereof is delivered to the subject at least once a week.

12. The method of claim 1, further comprising administering a therapeutically effective amount of an anti-cancer agent or anti-tumor agent to the subject, wherein the anti-cancer agent or anti-tumor agent is selected from the group consisting of a chemotherapeutic agent, an immunotherapeutic agent, a cytokine, an anti-angiogenic agent, a tyrosine kinase inhibitor (TKI), an apoptosis-inducing agent, an anti-cancer antibody, and combinations thereof.

13. (canceled)

14. The method of claim 1, further comprising administering an additional therapy, wherein the additional therapy is selected from the group consisting of an anti-tumor therapy, an anti-cancer therapy, a chemotherapy, a targeted therapy, an immunotherapy, a radiation therapy, a radiofrequency ablation therapy, surgery, a therapy using a tumor treating fields (TTFields) device, or combinations thereof.

15. A pharmaceutical composition for treating a cancer or a tumor in a subject comprising a therapeutically effective amount of 2,4-disulfonyl a-phenyl tert-butyl nitrone (2,4-ds-PBN) or pharmaceutically acceptable salts thereof, wherein the therapeutically effective amount is present in an amount to decrease a growth of the cancer or the tumor.

16. The pharmaceutical composition of claim 15, wherein the pharmaceutical composition is in a form of a tablet, a pill, a capsule, a gel, a liquid, a syrup, a slurry, or a suspension for an oral administration or an enteral administration.

17. The pharmaceutical composition of claim 16, wherein the therapeutically effective amount of the 2,4-ds-PBN or pharmaceutically acceptable salts thereof is from about 5 mg/kg body weight/day to about 1,000 mg/kg body weight/day.

18. The pharmaceutical composition of claim 15, wherein the composition comprises at least one coating layer, wherein the at least one coating layer encapsulates the 2,4-ds-PBN or pharmaceutically acceptable salts thereof and solubilizes at a predetermined range of pH.

19. The pharmaceutical composition of claim 15, wherein the pharmaceutical composition is in a form of a solution or a liquid for an intravenous injection or an intra-arterial injection.

20. The pharmaceutical composition of claim 19, wherein the therapeutically effective amount of the 2,4-ds-PBN or pharmaceutically acceptable salts thereof is from about 10 mg/kg body weight/day to about 500 mg/kg body weight/day.

21. The pharmaceutical composition of claim 15, wherein the cancer is a non-small cell lung carcinoma.
